(19) United States
(12) Patent Application Publication
(10) Pub. No.: US 2010/0035321 A1
(43) Pub. Date: Feb. 11, 2010

(54) SYSTEMS, DEVICES, AND, METHODS FOR RELEASING BIOMASS CELL COMPONENTS

(75) Inventors: Brian D. Wilkerson, Bellevue, WA (US); James A. Burns, JR., Seattle, WA (US); James C. Chen, Clyde Hill, WA (US)

Correspondence Address: SEED INTELLECTUAL PROPERTY LAW GROUP PLLC, 701 FIFTH AVE, SUITE 5400, SEATTLE, WA, 98104, US

(73) Assignee: Bionavitas, Inc., Redmond, WA (US)

(21) Appl. No.: 12/104,172

(22) Filed: Apr. 16, 2008

Related U.S. Application Data
(60) Provisional application No. 60/913,249, filed on Apr. 20, 2007.

(51) Int. Cl. C12N 13/00; C12M 1/00; C12N 1/12

(52) U.S. Cl. 435/173.1; 435/292.1; 435/257.1

(57) ABSTRACT

Systems, devices, and methods for releasing one or more cell components from a photosynthetic organism. A bioreactor system is operable for growing photosynthetic organisms. Some of the methods include contacting the photosynthetic organism with an energy-activatable sensitizer, and activating the energy-activatable sensitizer, thereby releasing a cellular component from at least one of, for example, a membrane structure, tubule, vesicle, cisterna, organelle, cell compartment, plastid, or mitochondrion, associated with the photosynthetic organisms.

### CROSS-REFERENCE TO RELATED APPLICATION

This application claims the benefit under 35 U.S.C. §119(e) of U.S. Provisional Patent Application No. 60/913,249 filed Apr. 20, 2007, the content of which is incorporated herein by reference in its entirety.

### BACKGROUND

1. Field

This disclosure generally relates to the field of molecular biology and microbiology and, more particularly, to systems, devices, and methods for releasing biomass cell components such as, for example lipids, proteins, vitamins, fatty acids, minerals, carotenoids, pigments, and the like.

2. Description of the Related Art

Biomasses such as, for example, mammalian, animal, plant, and insect cells, as well as various species of bacteria, algae, plankton, and protozoa, have many beneficial and commercial uses. For example, algal biomasses are used in wastewater treatment facilities to capture fertilizers, as carbon dioxide uptake agents, and as pollution control agents. Algal biomasses are also used to make biofuels. Likewise algal biomass cell components (e.g., lipids, proteins, vitamins, fatty acids, minerals, carotenoids, pigments, and the like) many also have beneficial and commercial uses including, for example, as pigmentation agents, nutritional supplements, energy sources, and pharmaceuticals.

A variety of methods and technologies exist for extracting biomass cell component including, for example, organic solvent extraction processes, maceration processes, and chromatography to name a few. Inefficient recovery of cell components, however, hampers many of these techniques.

Commercial acceptance of biomass products is dependent on a variety of factors such as, for example, cost to manufacture, cost to operate, reliability, durability, and scalability. Commercial acceptance of biomass products is also dependent on the ability to increase biomass product recovery, while decreasing biomass production cost. Therefore, it may be desirable to have novel approaches for harvesting biomass products including, for example, cell components such as lipids, proteins, vitamins, fatty acids, minerals, carotenoids, pigments, and the like.

The present disclosure is directed to overcome one or more of the shortcomings set forth above, and provide further related advantages.

### BRIEF SUMMARY

In one aspect, the present disclosure is directed to a method for releasing a cell component from a photosynthetic organism. The method includes contacting the photosynthetic organism with an energy-activatable sensitizer. In some embodiments, the energy-activatable photosensitizer is activatable by absorption of light (photosensitizer), sonic, ultrasonic, thermal, and/or chemical energy. The method may further include activating the energy-activatable photosensitizer, thereby releasing a cellular component from at least one of a membrane structure, tubule, vesicle, cisterna, organelle, cell compartment, plastid, or mitochondrion, associated with the photosynthetic organisms.

In some embodiments, the method includes recovering the cultivation media comprising the one or more cell components.

In another aspect, the present disclosure is directed to a system for releasing a cellular component of a photosynthetic organism. The system includes a bioreactor having a container, a first lighting system, and optionally cultivation media. In some embodiments, the container includes an exterior surface and an interior surface. The interior surface defines an isolated space configured to retain a plurality of photosynthetic organisms and cultivation media.

In some embodiments, the first lighting system comprising one or more energy-emitting substrates received in the isolated space of the container. Each of the energy-emitting substrates may include a first surface and a second surface opposite to the first surface. In some embodiments, the one or more energy-emitting substrates are configured to supply a first amount of energy from the first surface and a second amount of energy from the second surface to at least some of a plurality of photosynthetic organisms retained in the isolated space. In some embodiments, the first lighting system is operable to selectively emit energy having a peak emission wavelength ranging from about 400 nm to about 780 nm during a first period of time, and operable to selectively emit energy having a peak emission wavelength ranging from about 200 nm to about 400 nm during a second period of time, different than the first.

The system may optionally include cultivation media, retained in the isolated space, for sustaining a plurality of photosynthetic organisms. The cultivation media may further include at least one energy-activatable sensitizer such as a photosensitizer.

In another aspect, the present disclosure is directed a composition for releasing one or more growth factors, amino acids, nucleic acids, carotenoids, bioflavinoids, carbohydrates, chlorophylls, enzymes and co-enzymes, fatty acids, lipids, minerals, nucleic acids, pigments, proteins, and/or vitamins from an algal biomass into a collection medium. The composition includes a plurality of energy-activatable sensitizers and a permeabilizer. In some embodiments, the energy-activatable sensitizers are activatable by absorption of light, sonic, ultrasonic, thermal, and/or chemical energy. The permeabilizer allows absorption of the energy-activatable sensitizers by the photosynthetic biomass.

In yet another aspect, the present disclosure is directed to a process for producing and recovering one or more cell components from culture media including a plurality of photosynthetic organisms. The process includes inducing a change in the dielectric environment in the culture media and recovering the cultivation media comprising the one or more cell components.

In some embodiments, the method includes inducing a dielectric change that is sufficient to induce the photo-oxidative stress of a substantial portion of the plurality of photosynthetic organisms.

### BRIEF DESCRIPTION OF THE SEVERAL VIEWS OF THE DRAWING(S)

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements, as drawn, are not intended to convey any information regarding the actual shape of the particular elements, and have been solely selected for ease of recognition in the drawings.

FIG. 1 is a top front isometric view of a system to harvest cell component of photosynthetic organisms according to one illustrated embodiment.

FIG. 2 is a functional block diagram showing a system to harvest cell component of photosynthetic organisms according to one illustrated embodiment.

FIG. 3 is an exploded view of a bioreactor according to one illustrated embodiment.

FIG. 4 is a flow diagram of a method for releasing a cell component from a photosynthetic organism according to one illustrated embodiment.

FIG. 5 is a flow diagram for a process for producing and recovering one or more cell components from culture media including a plurality of photosynthetic organisms according to one illustrated embodiment.

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with bioreactors, the transmission of effluent streams into and out of a bioreactor, the photosynthesis and lipid extraction processes of various types of biomass (e.g., algae, and the like), fiber optic networks to include optical switching devices, light filters, solar collector systems to include solar array cells and solar collector mechanisms, methods of monitoring and harvesting a biomass (e.g., algae, and the like) to extract oil for biofuel purposes and/or convert a treated biomass (e.g., algae, and the like) to feedstock may not have been shown or described in detail to avoid unnecessarily obscuring the description.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “in another embodiment” means that a particular referent feature, structure, or characteristic described in connection with the embodiment is included in at least one embodiment. Thus, the appearance of the phrases “in one embodiment,” or “in an embodiment,” or “in another embodiment” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to a bioreactor system including “an energy-emitting substrate” includes a single energy-emitting substrate, or two or more energy-emitting substrates. It should also be noted that the term “or” is generally employed in its sense including “and/or” unless the content clearly dictates otherwise.

In some embodiments, the term “sensitizer” or “energy-activatable sensitizer” generally refers to a substance (e.g., chemical substances, energy activatable agents, photosensitizer agents, compounds, chemical entities, photosensitive chemicals, and the like) that upon absorption of energy (e.g., light, sonic, ultrasonic, thermal, and/or chemical energy, and the like) induces a chemical and/or physical alteration of another substance. In some embodiments, the sensitizer comprises a compound that is absorbed by, or preferentially associates with, one or more types of selected target biomasses, and, when exposed to energy of an appropriate waveband, absorbs the energy, causing a substances to be produced that chemical and/or physical alters the target biomass, or portions thereof.

Exemplary photosensitizers include aminolevulinic acid, bacteriochlorins, bacteriochlorophylls, benzoporphyrin derivatives, chlorins, indocyanine green, LUTRIN™ (lutetium texaphyrin, brand; Pharmacyclics, Inc., Sunnyvale, Calif.), merocyanines, methylene blue, myoglobin, catalase, cytochomes, phthalocyanines, porfimer sodium, hydro-mono benzoporphyrins, benzoporphyrin derivatives, porphyrins, porphyrin derivatives (e.g., protoporphyrin IX), pro-drugs such as delta-aminolevulinic acid that may produce photosensitive agents such as protoporphyrin IX, psoralens, purpurins, tetrapyrroles, texaphyrins, toluidine blue, nanoparticles including inorganic oxide-, metallic-, and polymer-based nanocomposites as photosensitizer carriers, and the like, or combinations thereof. In some embodiments, photosensitizers that absorbs light in a range of 500 nm 1100 nm.

Exemplary energy-activatable sensitizers include compounds that when exposed to energy of an appropriate waveband, absorbs the energy, causing substances the formation of radicals and/or singlet oxygen from triplet oxygen. In some embodiments, when activated, the energy-activatable sensitizers are capable of impairing or destroying target cells or biomass cell components in a biomass.

In some embodiments, the photosensitizers are capable of absorbing electromagnetic radiation, and are capable of catalyzing the formation of radicals and/or singlet oxygen from triplet oxygen under the influence of radiation.

The term “bioreactor” as used herein and the claims generally refers to any system, device, or structure capable of supporting a biologically active environment. Examples of bioreactors include fermentors, photobioreactors, stir-tank reactors, airlift reactors, pneumatically mixed reactors, fluidized bed reactors, fixed-film reactors, hollow-fiber reactors, rotary cell culture reactors, packed-bed reactors, macro and micro bioreactors, open containers, and the like, or combinations thereof.

In some embodiments, the bioreactor refers to a device or system for growing cells or tissues in the context of cell culture, such as the disposable chamber or bag, called a CELLBAG®, made by Panacea Solutions, Inc. and usable with systems developed by Wave Biotechs, LLC. In a further embodiment, the bioreactor can be a specially designed landfill for rapidly growing, transforming, and/or degrading organic structures. In yet a further embodiment, the bioreactor comprises a sphere and a mirror located outside of the sphere, wherein the shape of the sphere maximizes a surface to volume ratio of the algae contained therein and a waveguide for proving light from a light source, such as sunlight, into the sphere.

In some embodiments, two or more bioreactors may be coupled to form a multi-reactor system. In further embodiments, two or more bioreactors may be coupled in parallel and/or in series.

The term “biomass” as used herein and the claims generally refers to any biological material. Examples of a “biomass” include photosynthetic organisms, living cells, biological active substances, plant matter, living and/or recently living biological materials, and the like. Further examples of a “biomass” include mammalian, animal, plant, and insect cells, as well as various species of bacteria, algae, plankton, and protozoa.

The exemplary algae may include a taxonomically diverse group of organisms, typically found in most aquatic environments, including marine, freshwater, estuarine, and brackish water. Exemplary algae are also found in extreme environments (e.g., high salinity, high/low temperature, high pressure environments, and the like), as well as outside of typical aquatic environments, such as on cave walls, sidewalks, and the like. Most algal species are eukaryotes, (with a major exception being cyanobacteria which are prokaryotes.) Accordingly, in some instanced the algae includes membrane bound organelles, such as mitochandria, nucleus, ribosomes, endoplasmic reticulum, plastids, vacuoles, and chloroplasts. In some embodiments, the biomass comprises one or more prokaryotic algae and eukaryotic algae.

Algae are autotrophic, meaning that they can produce their own energy source via, for example photosynthesis. Photosynthesis generally occurs in an organelle, known as the chloroplast. This membrane bound organelle houses the chlorophyll pigment, which utilizes light energy to facilitate the reduction of carbon dioxide to glucose.

Many other cell components exist within the algae. Exemplary cell components include amino acids (e.g., Arginine, Histidine, Isoleucine, Leucine, Lysine, Methionine, Phenylalanine, Threonine, Tryptophan, Valine, and the like) anti-oxidants, B-Complex, carotenoids (e.g., beta-carotene), bioflavinoids, carbohydrates, catalase, cellulose, chlorophyll (e.g., chlorophyll a and b), cysteine, enzymes and co-enzymes, fatty acids (e.g., Linoleic, Linoleic 6, 9, 12, Oleic, Palmitic, Palmitoleic, Palmitolinoleic, Stearic, and the like), free radical scavengers, glutathione, lipids, minerals (e.g., Boron, Calcium, Chlorine, Chromium, Cobalt, Copper, Fluorine, Germanium, Iodine, Iron, Magnesium, Manganese, Molybdenum, Nickel, Phosphorus, Potassium, Selenium, Silicon, Sodium, Titanium, Vanadium, Zinc, and the like), neuropeptide precursors, nucleic acids (e.g., deoxyribonucleic acids (DNA), ribonucleic acids (RNA) RNA, and the like), pigments, polygalactans, proteins (e.g., glyco-proteins), selenium, silica, superoxide dismutase, tetrapyrroles, vitamins (e.g., Ascorbic Acid C, Biotin, Choline, Cobalamin B12, Folic Acid, Niacin, Pantothenic Acid B5, Provitamin A Beta Carotene, Pyridoxine B6, Riboflavin B2, Thiamine B1, Vitamin E, and the like), other essential growth factors, and the like.

Membranes, in general, are not impermeable, but they are structured such that they regulate the passage of materials into and out of a cell or organelle. Membranes are composed of bilayers of phospholipids. Membranes may include associated proteins that can server the function of providing structural integrity, facilitating the uptake/secretion of ions, or catalyzing reactions among other tasks. These proteins may be present on a surface of the membrane, or may transverse the entire membrane.

Lipids are also utilized as energy storage compounds, usually in the form of triacylglycerols. Lipids in this form are less oxidized than other compounds and thus release more energy when oxidized during respiration.

A variety of methods and technologies exist for cultivating and harvesting biomasses such as, for example, mammalian, animal, plant, and insect cells, as well as various species of bacteria, algae, plankton, and protozoa. These methods and technologies include open-air systems and closed systems. Algal biomasses, for example, are typically cultured in open-air systems (e.g., ponds, raceway ponds, lakes, and the like). Alternatively, biomasses may be cultivated in closed systems called bioreactors.

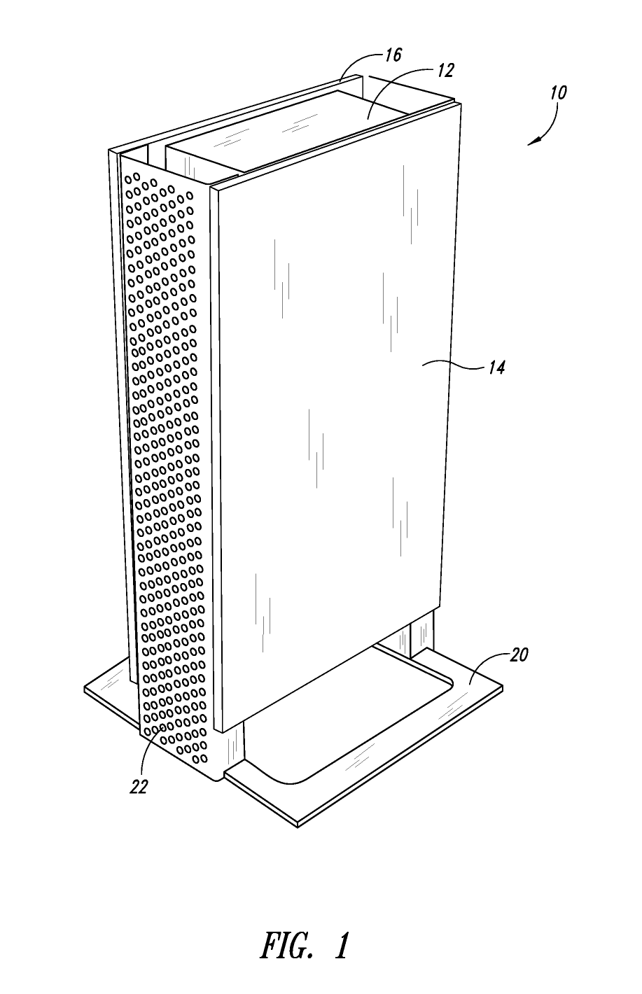

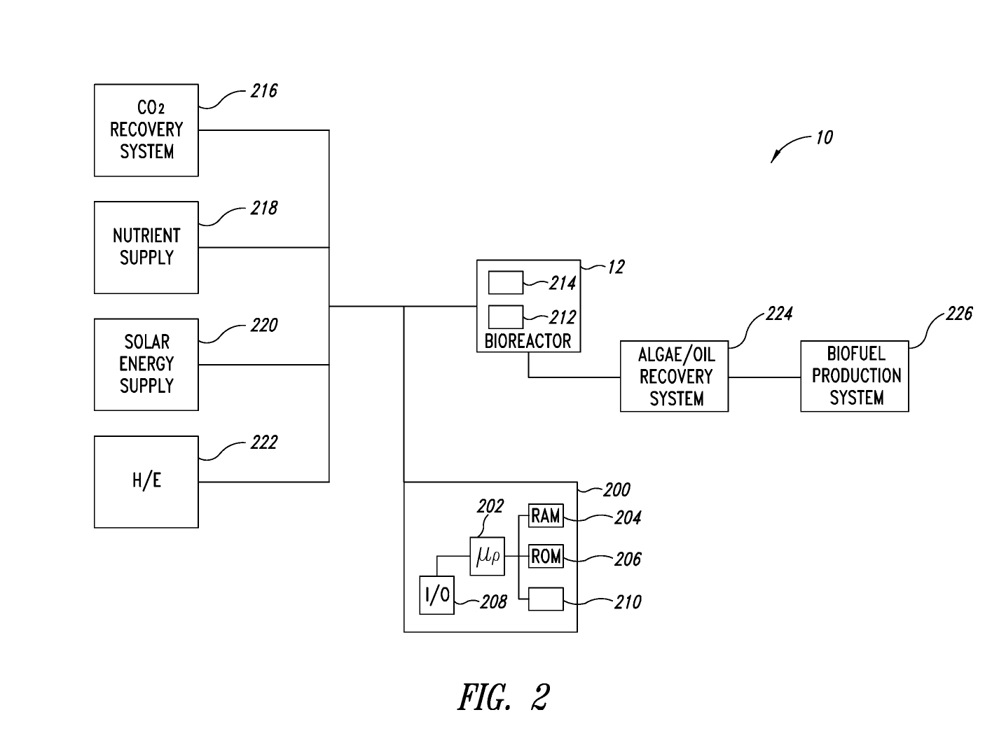

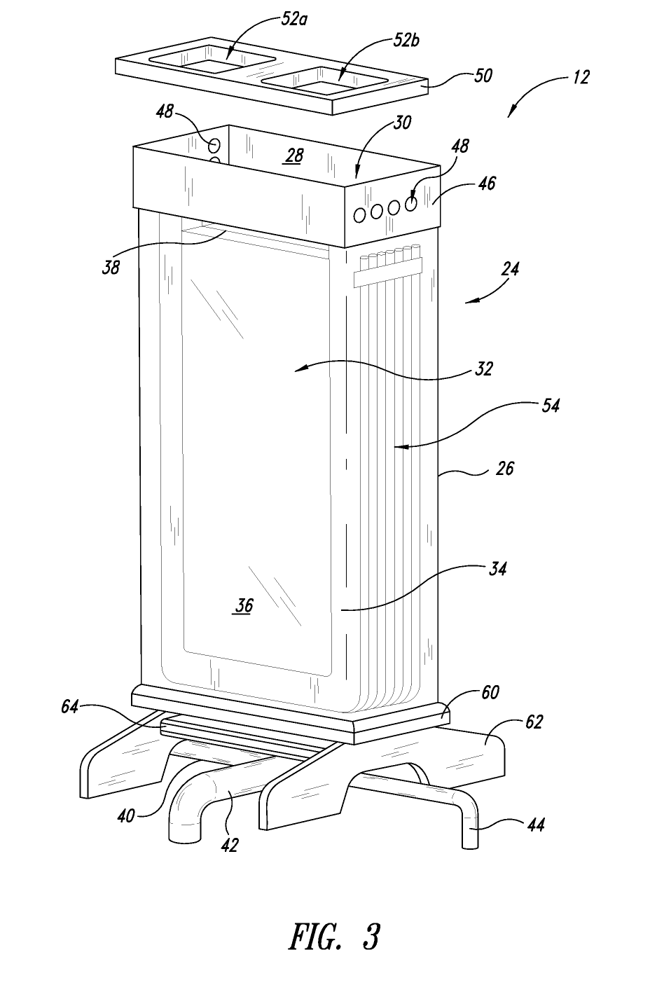

FIGS. 1, 2, and 3 show an exemplary system for releasing a cellular component of a photosynthetic organism. The system 10 includes a bioreactor 12, housing structures 14, 16, and a support structure 20. The system 10 may further include a side structure 22.

The system 10 may further include a control system 200 operable to control the voltage, current, and/or power delivered to the bioreactor 12, as well as automatically control at least one process variable and/or a stress variable that alters or affects the growth and/or development of an organism (e.g., changing stress variable to induce nutrient deprivation, nitrogen-deficiency, silicon-deficiency, pH, CO₂ levels, oxygen levels, degree of sparging, or other conditions that affect growth and/or development of an organism). In some embodiments, the bioreactor 12 may operate under strict environmental conditions that require controlling of one or more process variables associated with cultivating and/or growing a photosynthetic biomass. For example, the system 10 may include one or more sub-systems for controlling gas flow rates (e.g., air, oxygen, CO₂, and the like), effluent streams, temperatures, pH balances, nutrient supplies, other organism stresses, and the like.

The control system 200 may include one or more controllers 202, for example, microprocessors, digital signal processor (DSPs) (not shown), an application-specific integrated circuits (ASICs) (not shown), field programmable gate arrays (FPGAs) (not shown), and the like. The control system 200 may also include one or more memories, for example, random access memory (RAM) 204, read-only memory (ROM) 206, and the like, coupled to the controllers 202 by one or more busses. The control system 200 may further include one or more input devices 208 (e.g., a keypad, touch-screen display, and the like). The control system 200 may also include discrete and/or an integrated circuit elements 210 to control the voltage, current, and/or power. In some embodiments, the control system 200 is configured to control at least one of light intensity, illumination intensity, a light-emitting pattern, a peak emission wavelength, an on-pulse duration, and a pulse frequency associated with one or more energy-emitting substrates 34 based on a measured optical density.

The system 10 may further include a variety of controller systems 200, sensors 212, as well as mechanical agitiators 214, and/or filtration systems, and the like. These devices may be controlled and operated by a central control system 200. In some embodiments, the one or more sensors 212 may be operable and/or configured to determine at least one of a temperature, pressure, light intensity, optical density, opacity, gas content, pH, fluid level, sparging gas flow rate, salinity, fluorescence, absorption, mixing, and/or turbulence. The controller 200 may be configured to control at least one of an illumination intensity, illumination pattern, peak emission wavelength, on-pulse duration, and/or pulse frequency based on a sensed temperature, pressure, light intensity, optical density, opacity, gas content, pH, fluid level, sparging gas flow rate, salinity, fluorescence, absorption, mixing, and/or turbulence.

The system 10 may also include sub-systems and/or devices that cooperate to monitor and possibly control operational aspects such as the temperature, salinity, pH, CO₂ levels, O₂ levels, nutrient levels, and/or a light supply, and the like. In some embodiments, the system 10 may include the ability to increase or decrease each aspect or parameter individually or in any combination, for example, temperature may be raised or lowered, gas (e.g., CO₂, O₂, etc.) levels may be raised or lowered, pH, nutrient levels, light, may be raised or lowered. The light can be natural or artificial. Some general lighting control aspects include controlling the duration that the light operates on portions of, for example, an algal mass in the bioreactor 12, cycling the light (to include periods of light and dark), for example artificial light, to extend the growth of the algae past daylight hours, controlling the wavelength of the light, controlling the lighting patterns, and/or controlling the intensity of the light. Lighting control may also include controlling one or more filters, operatives, masks, shades, and/or levers, particularly where the light is natural.

The system 10 may further include a carbon dioxide recovery system 216 for recovering, treating, extracting, utilizing, scrubbing, cleaning, and/or purifying a carbon dioxide supply from, for example, flue gas of an industrial source (e.g., an industrial plant, an oil field, a coal mine, and the like).

The system 10 may further include one or more nutrients supply systems 218, solar energy supply systems 220, and heat exchange systems 222.

The nutrients supply systems 218 may include, or be part of, one or more effluent and/or nutrient streams. An effluent is generally regarded a something that flows out or forth, like a stream flowing out of a body of water. For example, this includes, but is not limited to discharge wastewater from a waste treatment facility, brine wastewater from desalting operations, and/or coolant water from a nuclear power plant. In the context of algae cultivation, an effluent stream contains nutrients to feed algae present inside and/or outside of a bioreactor 12. In one embodiment, the effluent stream includes biological waste or waste sludge from a waste treatment facility (e.g., sewage, landfill, animal, slaughterhouse, toilet, outhouse, portable toilet waste, and the like). Such an effluent stream (including the CO₂ produced by the bacteria within such waste) can be directed to the algae, where the algae remove nitrogen, phosphate, and carbon dioxide (CO₂) from the stream. In another embodiment, the effluent stream comprises flue gases from power plants. The algae remove the CO₂ and various nitrogen compounds (NOx) from the flue gases. In each of the foregoing embodiments, the algae use the CO₂, in particular, for the process of photosynthesis. The oxygen produced by the algae during the photosynthetic process could be utilized to promote, for example, further bacterial growth and CO₂ production in a waste effluent stream. Furthermore, it is understood that the effluent streams can be seeded with a variety of additional nutrients and/or biological material to stimulate and enhance the growth rate, photosynthetic process, and overall cultivation of the algae.

The solar energy supply systems 220 may collect and/or supply sunlight, as well as direct light into the bioreactor 12. In some embodiments, the solar energy supply systems 220 includes a solar energy collector and a solar energy concentrator including a plurality of optical elements configured and positioned to collect and concentrate sun light. In some embodiments, the solar energy supply systems 220 is operable to selectively emit energy having a peak emission wavelength ranging from about 400 nm to about 780 nm during a first period of time, while selectively preventing the emission of energy having a peak emission wavelength ranging from about 200 nm to about 400 nm. In some embodiments, the solar energy supply systems 220 is operable to selectively emit energy having a peak emission wavelength ranging from about 200 nm to about 400 nm during a second period of time, different than the first.

The heat exchange system 222 typically controls and/or maintains a constant temperature within the bioreactor 12. For example, temperature may be lowered to stress the algae to promote oil production, etc. at end of growth cycle. In some embodiments, the heat exchange system 222 and the control system 200 operate to maintain a constant temperature in the bioreactor 12 to sustain a bioprocess within.

In some embodiments, the system 10 may further include a supply systems for introducing photosensitzers to algae present inside and/or outside of a bioreactor 12. For example, a supply systems for introducing photosensitzers to a mixed culture of one or more species of algae.

The bioreactor 12 may include at least one container 24 having and exterior surface 26 and an interior surface 28. In some embodiments, the interior surface 28 defines an isolated space 30 configured to retain biomasses, photosynthetic organisms, living cells, biological active substances, and the like. For example, the isolated space 30 defined by the interior surface 28 of the container 24 may be used to retain a plurality of photosynthetic organisms and cultivating media. The isolated space 30 can be a reservoir or collection region for holding biomass-producing material.

The bioreactor 12 may take a variety of shapes, sizes, and structural configurations, as well as comprise a variety of materials. For example, the bioreactor 12 may take a cylindrical, tubular, rectangular, polyhedral, spherical, square, pyramidal shape, and the like, as well as other symmetrical and asymmetrical shapes. In some embodiments, the bioreactor 12 may comprise a cross-section of substantially any shape including circular, triangular, square, rectangular, polygonal, and the like, as well as other symmetrical and asymmetrical shapes. In some embodiments, the bioreactor 12 may take the form of an enclosed vessel 32 having one or more enclosures and/or compartments capable of sustaining and/or carrying out a chemical process such as, for example the cultivation of photosynthetic organisms, organic matter, a biochemically active substances, and the like.

Among the materials useful for making the container 24 of the bioreactor 12 examples include, translucent and transparent materials, optically conductive materials, glass, plastics, polymer materials, and the like, or combinations or composites thereof, as well as other materials such as stainless steel, Kevlar, and the like, or combinations or composites thereof.

In some embodiments, the container 24 may comprise one or more transparent or translucent materials to allow light to pass from the exterior surface to a plurality of photosynthetic organisms and cultivation media retained in the isolated space 30. In some further embodiments, a substantial portion of the container 24 comprises a transparent or translucent material. Examples of transparent or translucent materials include glasses, PYREX® glasses, plexiglasses, acrylics, polymethacrylates, plastics, polymers, and the like or combinations or composites thereof.

The bioreactor 12 may also include a first lighting system 32. In some embodiments, the first lighting system 32 is received in the isolated space 30 of the container 24. The first lighting system 32 may comprise one or more energy-emitting substrates 34. In some embodiments, the energy-emitting substrates 34 take the form of light-emitting substrates.

In some embodiments, each energy-emitting substrate 34 has a first surface 36 and a second surface 38 opposite to the first surface. The one or more energy-emitting substrates 34 may supply a first amount of light from the first surface 36 and a second amount of light from the second surface 38 to at least some of a plurality of photosynthetic organisms retained in the isolated space 30. In some embodiments, the one or more energy-emitting substrates 34 are configured to provide at least a first and a second energy-emitting pattern. The first lighting system 32 may further operate to produce at least a first illumination intensity level and a second illumination intensity level different than the first. In some embodiments, the second amount of light has at least one characteristic (e.g., light intensity, illumination intensity, light-emitting pattern, peak emission wavelength, on-pulse duration, and/or pulse frequency) different than a like characteristic of the first amount of light. In some other embodiments, the second amount of light has the same characteristics as the first amount of light.

In some embodiments, the bioreactor 12 may include one or more mirrored and/or reflective surfaces received in and/or formed on the interior 30 of the bioreactor 12. In some embodiments, a portion of the interior surface 28 of the bioreactor 12 may include mirrored and/or reflective surfaces such as, for example, a film, coating, optically active coating, mirrored and/or reflective substrate, and the like. In some embodiments, the housing structures 14, 16 may include one or more mirrored and/or reflective surfaces in a portion adjacent to the exterior surface 26 of the container 24.

In some embodiments, the one or more mirrored and/or reflective surfaces may be configured to maximize distribution of light emitted by a lighting system 32.

The energy-emitting substrates 34 may comprise a single energy-emitting surface (e.g., a single light-emitting surface), or may comprise a multi-side arrangement with a plurality of energy-emitting surfaces. The energy-emitting substrates 34 may come in a variety of shapes and sizes. In some embodiments, the energy-emitting substrates 34 may comprise a cross-section of substantially any shape including circular, triangular, square, rectangular, polygonal, and the like, as well as other symmetrical and asymmetrical shapes.

The one or more energy-emitting substrates 34 may include a plurality of light emitting diodes (LEDs). LEDs including organic light-emitting diodes (OLEDs) come in a variety of forms and types including, for example, standard, high intensity, super bright, low current types, and the like. The “color” and/or peak emission wavelength spectrum of the emitted light generally depends on the composition and/or condition of the semi-conducting material used, and may include peak emission wavelengths in the infrared, visible, near-ultraviolet, and ultraviolet spectrum. Typically the LEDs' color is determined by the peak wavelength of the light emitted. For example, red LEDs have a peak emission ranging from about 625 nm to about 660 nm. Examples of LEDs colors include amber, blue, red, green, white, yellow, orange-red, ultraviolet, and the like. Further examples of LEDs include bi-color, tri-color, and the like. Emission wavelength may also depend on current delivered to the LEDs.

Certain biomasses, for example plants, algae, and the like comprise two types of chlorophyll, chlorophyll a and b. Each type typically possesses a characteristic absorption spectrum. In some cases, the spectrum of photosynthesis of certain biomasses is associated with (but not identical to) the absorption spectra of, for example, chlorophyll. For example, the absorption spectra of Chlorophyll a may include absorption maxima at about 430 nm and 662 nm, and the absorption spectra of Chlorophyll b may include absorption maxima at about 453 nm and 642 nm. In some embodiments, the one or more energy-emitting substrates 34 may be configured to provide one or more peak emissions associated with the absorption spectra of chlorophyll a and chlorophyll b.

The plurality of LEDs may take the form of, for example, at least one LED array. In some embodiments, the plurality of LEDs may take the form of a plurality of two-dimensional LED arrays or at least one three-dimensional LED array.

The array of LEDs may be mounted using, for example, a flip-chip arrangement. A flip-chip is one type of integrated circuit (IC) chip mounting arrangement that does not require wire bonding between chips. Thus, wires or leads that typically connect a chip/substrate having connective elements can be eliminated to reduce the profile of the one or more energy-emitting substrates 34.

In some embodiments, instead of wire bonding, solder beads or other elements can be positioned or deposited on chip pads such that when the chip is mounted upside-down in/on the energy-emitting substrates 34, electrical connections are established between conductive traces of the energy-emitting substrates 34 and the chip.

In some embodiments, the plurality of LEDs comprise a peak emission wavelength ranging from about 440 nm to about 660 nm, an on-pulse duration ranging from about 10 μs to about 10 s, and a pulse frequency ranging from about 1 μs to about 10 s.

In some embodiments, the one or more energy-emitting substrates 34 include a plurality of optical waveguides to provide optical communication between a source of light located in the exterior of the bioreactor 12 and a portion of the first lighting system 32 received in the isolated space 30. In some embodiments, the optical waveguides take the form of a plurality of optical fibers.

In some embodiments, the first lighting system 32 may further include at least one optical waveguide on the exterior surface 26 of the container 24 optically coupled to the first lighting system 32. The at least one optical waveguide may be configured to provide optical communication between a source of solar energy and a portion of the first lighting system 32 received in the isolated space 30. The source of solar energy may include a solar collector and a solar concentrator optically coupled to the solar collector and the portion of the first lighting 32. The solar concentrator can be configured to concentrated solar energy provided by the solar collector and to provide the concentrated solar energy to the portion of the first lighting system 32 received in the isolated space 30.

In some embodiments, the one or more energy-emitting substrates 34 are encapsulated in a medium having a first index (n₁) of refraction and the growth medium has a second index of refraction (n₂) such that the differences between n₁ and n₂, at a given wavelength selected from a spectrum ranging from about 440 nm to about 660 nm, is less than about 1. Examples of the medium having a first index (n₁) of refraction include mineral oil. Mineral oil may also serve to cool the LEDs and prevent water migration into the electronics, for instance in the event of a panel case seal failure.

In some embodiments, the control system 200 is configured to control at least one of a light intensity, illumination intensity, energy-emitting pattern, peak emission wavelength, on-pulse duration, and/or pulse frequency associated with the energy-emitting substrates 34 based on a measured optical density.

The one or more energy-emitting substrates 34 may be configured to supply an effective amount of light to a substantial portion of the plurality of photosynthetic organisms retained in the isolated space 30. In some embodiments, an effective amount of light comprises an amount sufficient to sustain a biomass concentration having an optical density (OD) value greater than from about 0.1 g/l to about 15 g/l. Optical density may be determined by having an LED on the surface of one panel and an optical sensor directly opposite on the surface of another panel. Alternatively, the initial sensor may be a separate device inside the medium. For each algae species, samples of the growth are taken and a concentration level is determined by filtering the algae and weighing the results. Samples are taken at a minimum of three different concentration levels and those values are corresponded to the optical readings from between the panels or device inside the medium and an algorithm is created using the data. Optical density may then be monitored optically and manipulated with the control system 200.

In some further embodiments, an effective amount of light comprises an amount sufficient to activate a substantial portion of a plurality of energy-activatable photosensitizers included in a volume of cultivation media comprising a biomass.

In some embodiments, an effective amount of light comprises an amount sufficient to sustain a photosynthetic organism density greater than 1 gram of photosynthetic organism per liter of cultivation media. In some embodiments, an effective amount of light comprises an amount sufficient to sustain a photosynthetic organism density greater than 5 grams of photosynthetic organism per liter of cultivation media. In some further embodiments, an effective amount of light comprises an amount sufficient to sustain a photosynthetic organism density ranging from about 1 gram of photosynthetic organisms per liter of cultivation media to about 15 grams of photosynthetic organisms per liter of cultivation media. In yet some other embodiments, an effective amount of light comprises an amount sufficient to sustain a photosynthetic organism density ranging from about 10 grams of photosynthetic organisms per liter of cultivation media to about 12 grams of photosynthetic organisms per liter of cultivation media. In some embodiments, the bioreactor 12 may further include conductivity probe 70. The system 10 may further include one or more sensors including dissolved oxygen sensors 72, 74, pH sensors 76, 78, a level sensor 68, CO₂ sensors, oxygen sensors, and the like. The system 10 may also include one or more thermocouples 6. The bioreactor 12 may include, for example, inlet and/or outlet ports 48, and inlet and/or outlet conduits 40, 42, 44, for providing or discharging process elements, nutrients, gasses, biomaterials, and the like, to and from the bioreactor 12.

Growth media may be for freshwater, estuarine, brackish, or marine bacterial or algal species and/or other microorganisms or plankton. The growth media may consist of salts, such as sodium chloride and/or magnesium sulfate, macro-nutrients, such as nitrogen and phosphorus containing compounds, micro-nutrients such as trace metals, for example iron and molybdenum containing compounds and/or vitamins, such as Vitamin B12. The growth media may be modified or altered to accommodate various species and/or to optimize various characteristics of the cultured species, such as growth rate, protein production, lipid production, and carbohydrate production. In some embodiments, the growth media may include one or more photosensitizers.

The system 10 may further include a second lighting system adjacent to the exterior surface 26 of the container. The second lighting system may comprise at least one energy-emitting substrate 34 configured to provide light to at least some of the plurality of photosynthetic organisms retained in the isolated space 30 and located proximate to a portion of the interior surface 26 of the container 24. In some embodiments, the second lighting system includes at least one energy-emitting substrate located on one side of housing structure 14, and at least one energy-emitting substrate located on one side of housing structure 16.

In some embodiments, the one or more energy-emitting substrates 34 take the form of light-energy-supplying substrates 34a having a first side 92 and a second side 94 opposite to the first side 92, the first and the second sides 92, 94 including one or more light-energy-supplying elements 92 that form part of a light-energy-supplying area 96. In some embodiments, each of the light-energy-supplying substrates 34a may be encapsulated, covered, laminated, and/or included in a medium having a first index (n₁) of refraction and the cultivation media has a second index of refraction (n₂) such that the differences between n₁ and n₂, at a given wavelength selected from a spectrum ranging from about 440 nm to about 660 nm, is less than about 1.

In some embodiments, the light-energy-supplying substrates 34a include a plurality of light sources 92 mounted to a flexible transparent base that forms part of the light-energy-supplying area 96. The light sources 92 can be wire bonded or mounted in a flip chip arrangement onto the flexible transparent base. In some embodiments, the light-energy-supplying substrates 34a may include a plurality of optical waveguides to provide optical communication between a source light located in the exterior of the bioreactor 12 and the plurality of light-energy-supplying substrates received within the isolated space 30 of the bioreactor 12. In some embodiments, the energy-emitting substrates 34 may be porous and hydrophilic.

In some embodiments, the system 10 may take the form of a photosynthetic biomass cultivation system. The biomass cultivation system includes a control system 200 configured to automatically control at least one process variable associated with cultivating a photosynthetic biomass, and a bioreactor 12. The bioreactor 12 includes a structure 24 and a lighting system 32.

The structure 24 includes an exterior surface 26 and an interior surface 28, the interior surface 28 defines an isolated space 30 comprising a volume configured to retain the photosynthetic biomass suspended in cultivation media. The lighting system 32 is received in the isolated space 30 of the structure 24. In some embodiments, the lighting system 32 includes one or more energy-emitting elements 34 including a light-emitting area 96 on each side of it sides 94, 98. The light-emitting area 96 forms part of a light-emitting-area 96 to reactor-volume interface. In some embodiments, the energy-emitting area to bioreactor volume ratio ranges from about 0.005 m²/L to about 0.1 m²/L. The energy-emitting elements may take the form of a plurality of two-dimensional LED arrays or at least one three-dimensional LED array.

The photosynthetic biomass cultivation system may include one or more sensors 212 operable to determine at least one of a temperature, pressure, light intensity, density, gas content, pH, fluid level, sparging gas flow rate, salinity, fluorescence, absorption, mixing, turbulence and/or the like.

The control system 200 is configured to automatically control the at least one process variable selected from a bioreactor interior temperature, bioreactor pressure, pH level, nutrient flow, cultivation media flow, gas flow, carbon dioxide gas flow, oxygen gas flow, light supply, and/or the like.

In some embodiments, the bioreactor 12 comprises one or more effluent streams providing fluidic communication of gasses, liquids, and the like between the exterior and/or interior of the bioreactor 12. In some embodiments, the bioreactor 12 make take the form of enclosed system wherein no effluent streams go in or out on a continual basis.

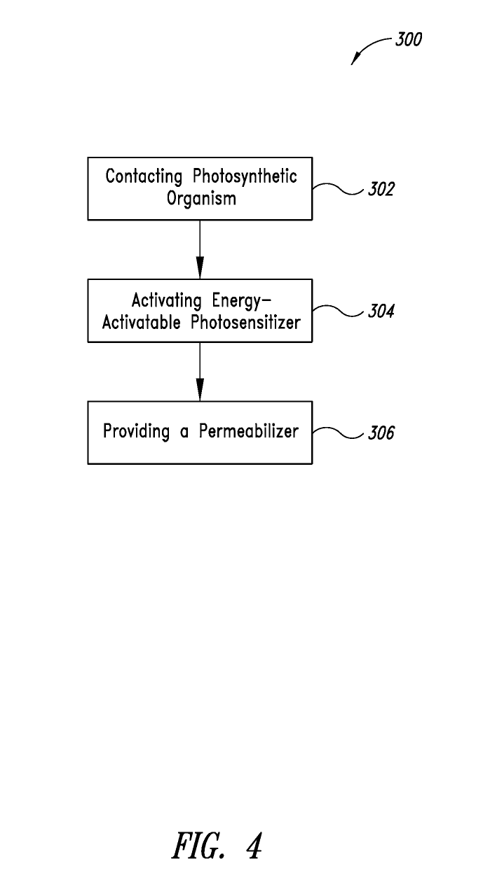

FIG. 4 shows an exemplary method 300 for releasing a cell component from a photosynthetic organism.

At 302, the method 300 includes contacting the photosynthetic organisms and cultivation media with a composition including a plurality of energy-activatable photosensitizers, the energy-activatable photosensitizer activatable by absorption of light, sonic, ultrasonic, thermal, and/or chemical energy.

In some embodiments, the energy-activatable photosensitizer, when activated, is capable of disrupting, rupturing, degrading, and/or breaking the cell wall, and/or the cell membrane. In some embodiments, the energy-activatable photosensitizer, when activated, is capable of disrupting, rupturing, degrading and/or breaking the membranes of organelles. In some embodiments, the energy-activatable photosensitizer, when activated, is capable of disrupting, rupturing, degrading and/or breaking the cell nucleus. In some embodiments, the energy-activatable photosensitizer, when activated, is capable of disrupting and/or lysing cells in a culture or a concentrate. In some embodiments, photosensitizers may be used to liberate proteins or other non-lipid material from previously disrupted and/or lysed cells in culture or in concentrate.

In some embodiments, photosensitizers may be used to liberate chloroplasts and/or chlorophyll from a lipid extract obtained by organic or physical extraction. In some embodiments, photosensitizers may be used to liberate organelles or other cell components from a lipid extract obtained by organic or physical extraction. In some embodiments, photosensitizers may be used to liberate lipids utilized as cellular carbon reserve materials, or liberate lipids utilized as structural components of membranes. In some embodiments, photosensitizers may be used to degrade the non-lipid components of organelles to facilitate collection of lipids contained within the organelle or lipids in the organelle membrane.

In some embodiments, the photosensitizers may be selectively targeted using, for example, a targeting moiety that targets a particular type of cell, a particular region of the cell, or a particular component of the cell.

The term “targeting moiety” refers to any molecular structure which assists a substance, compound, or other molecule in binding or otherwise localizing to a particular target, a target area, entering target cell(s), binding to a target receptor, and the like. For example, targeting moieties may comprise peptides, lipids (e.g., cationic, neutral, and steroidal lipids, virosomes, and liposomes), antibodies, lectins, ligands, sugars, steroids, hormones, nutrients, proteins, and the like.

In some embodiments, the photosensitizers may be selectively targeted to a particular type of cell, a particular region of the cell, or a particular component of the cell by controlling an incubation time or a time of initiation of incubation.

In some embodiments, the photosensitizers may target a specific membrane bound protein or a general group of proteins, a specific membrane component (e.g., glycolipid, oligosaccharide, polysaccharide, and the like), or a general class of glycolipids, oligosaccharides, or polysaccharides. In some embodiments, the photosensitizers may be targeted to a particular region of the cell by controlling at least one of a temperature, salinity, dissolved oxygen level, carbon dioxide level, trace metals content, nitrogen compounds content, phosphorus compounds content, sodium salts content, calcium salts content, magnesium salts content, sulfates content, sulfides content, potassium salts content, or other algal medium components and parameters. In some embodiments, the photosensitizers may be targeted to a specific molecular structure, shape motif on the surface of the cell, or shape motif internal to the cell. In some further embodiments, the photosensitizers may be targeted to a general class of molecular structures, shapes motifs on the surface of the cell, or shapes motifs internal to the cell.

In some embodiments, the photosensitizers may be introduced to the surface of the cell or into the cell by controlling at least one of a temperature, salinity, pH, dissolved oxygen, carbon dioxide, trace metals, nitrogen compounds, phosphorus compounds, sodium salts, calcium salts, magnesium salts, sulfates, sulfides, potassium salts, or other algal medium components and parameters. In some embodiments, the photosensitizers can be added during a dark or a light period of the algal culture incubation, or may be added directly to the culture medium at any stage of the algal culture.

In some embodiments, two or more photosensitizers can be employed either simultaneously or in succession, or in conjunction with other chemicals or physical processes, to facilitate the collection and/or concentration of lipids or proteins or other value products.

In some embodiments of the disclosed systems, devices and methods, the lighting conditions may be operably controlled to favor, for example, growth conditions, lysing conditions, harvesting conditions, or combinations thereof. For example, in some embodiments, the first lighting system is operable to selectively emit energy having a peak emission wavelength ranging from about 400 nm to about 780 nm during a first period of time, while selectively preventing the emission energy having a peak emission wavelength ranging from about 200 nm to about 400 nm. In some embodiments, the first lighting system is operable to selectively emit energy having a peak emission wavelength ranging from about 200 nm to about 400 nm during a second period of time, different than the first.

In one embodiment of a photobioreactor utilizing solar energy directed into fiber optics, only photosynthetically active radiation (PAR) light is passed on to the growing algae. The UV and IR wavelengths are filtered out. Once the algae has completed its growth and is harvested the “wasted” UV light may be directed into the algae medium with a photosensitizer to create the desired membrane disruption (e.g., activation of the energy activatable photosensitizer).

Visible spectrum and UV-A light do not typically cause direct damage to plants and biological organisms. This effect changes, however, when in the presence of a kind of light-absorbing molecules called photosensitizers. Cells that have absorbed photosensitizers can be rapidly damaged or killed when exposed to UV-A or visible spectrum radiation. It is estimated that thousands of natural and synthetic molecules can function as photosensitizers. The excited sensitizer molecule can react directly with the mixture or with other molecules (frequently oxygen) in the reaction mixture, giving products that can react with the mixture.

In some instances, a photosensitizer, on absorption of a photon, promotes an electron to a higher energy state. Very few reactions occur during this singlet state because of its short lifetime. The singlet-excited state, however, can undergo a fast spin inversion to give a metastable triplet state. Triplet states typically have a much longer life span than the singlet state. This allows the triplet states to undergo a large number of collisions with other molecules and as a result are highly efficient at transferring energy. In most reactions, the triplet sensitizer returns to ground state and can absorb another photon.

During photosensitized reactions, photons are absorbed by the sensitizer molecule. The resulting energy-rich state then undergoes reactions that ultimately result in the chemical alteration of another molecule in the system. Some photosensitizers are very effective for substrate molecules in solution but are ineffective with cells because they do not generally penetrate into the cell.

Typically, photooxidation increases with increases in pH. In some embodiments, rates increase rapidly in the presence of pH ranging from a pH of about 7 up to about a maximum pH of about 10.5.

Phototoxicity is the general term used for damaging or killing cells by using photosensitized reactions. Cell membranes can act as a differential barrier to the penetration for photosensitizers into the cell. This process can be helped by utilizing permeabilizers. Because cells are made from many kinds of molecules possessing a variety of physical and chemical properties, photosensitizers can be targeted to one or more of the various components of the cell; thus, the product of the reaction can be tailored to isolate a desired product such as lipids or proteins.

Algae or other plants may be grown under controlled light conditions for example with exposure to 440 nm to 680 nm photons. The algae can be grown in the presence of a photosensitizer capable of being activated by energy having a wavelength outside of the photosynthetically active radiation range of about 400 to about 700, for example in the UV-A range. These methods may allow for the cultivation of the biomass in the presence of a photosensitizer.

In some embodiments, contacting the photosynthetic organisms and cultivation media with a composition including a plurality of energy-activatable photosensitizers, may include introducing energy-activatable photosensitizers to the photosynthetic organisms via a chemical, synthetic or biological vector. The cellular uptake of the photosensitizers may be facilitated by the use of a physical process and/or chemical to increase the permeability of the cell wall and/or cell membrane. In some embodiments, the cellular organelle uptake of the photosensitizers may be facilitated by the use of a physical process and/or chemical to increase permeability of the organelle membrane.

At 304, the method 300 may further include: releasing one or more of the cell components into the cultivation media by activating a substantial portion of the plurality of energy-activatable photosensitizers; and disrupting, with the activated energy-activatable photosensitizers, at least one of a membrane structure, tubule, vesicle, cisterna, organelle, cell compartment, plastid, or mitochondrion, associated with the photosynthetic organisms.

At 306, the method 300 includes recovering the cultivation media comprising the one or more cell components.

In some embodiments, recovering the cultivation media comprising the one or more cell components includes concentration the Algal cells by, for example, centrifugation, filtration, reverse filtration, evaporation, or other physical methods prior to addition of the photosensitizers.

Lipids may be concentrated by centrifugation, filtration, reverse filtration, evaporation, or other physical methods after the addition of photosensitizers.

The algal culture may be moved to a different holding tank and maintained under the same environmental parameters or altered parameters prior, during or after the addition of photosensitizers.

The algal culture may be moved to a different holding tank and maintained under the same environmental parameters or altered parameters prior, during or after the activation of a plurality of photosensitizers.

At 308, the method 300 may further include providing a permeabilizer to the photosynthetic organisms, the permeabilizer capable of promoting absorption of the energy-activatable photosensitizers by the photosynthetic organisms. Among permeabilizers, examples include dimethyl dulfoxide (DMSO), polyethylene-imine, lactic acid, and the like. Further examples of suitable permeabilizers are disclosed in, for example, PCT Publication No. WO/2003/101197 and PCT Publication No. WO/2004/091584. In some embodiments, a permeabilizer is used to promote photosensitizer absorption.

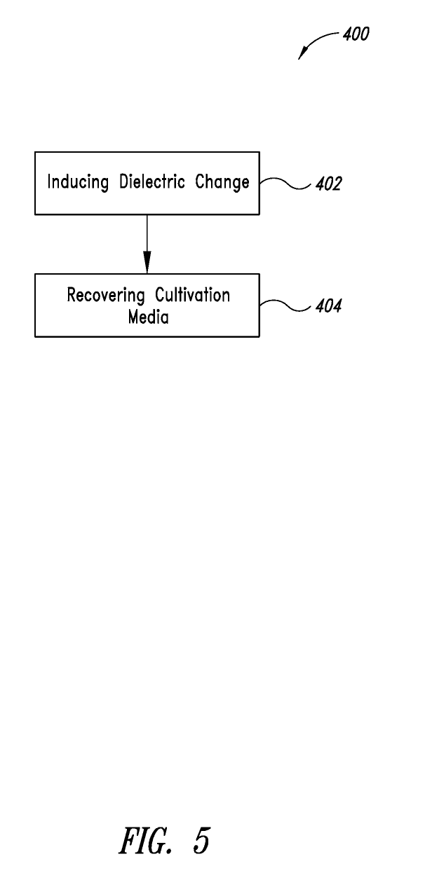

FIG. 5 shows an exemplary process 400 for producing and recovering one or more cell components from culture media including a plurality of photosynthetic organisms.

At 402, the process 400 includes inducing a dielectric change in the culture media, the induced dielectric change sufficient to induce photo-oxidative stress of a substantial portion of the plurality of photosynthetic organisms.

Changing the dielectric properties of the solution can have significant effects on the efficiency of the photooxidation. For example, plants contain different photosensitizers such as chlorophyll a and b. If the photosynthetic process is blocked by heat treatment or carbon dioxide starvation, illumination kills the photosynthetic tissues. In some embodiments, this provides a method for breaking down the cells for harvesting value cell components. In some embodiments, controlling temperature may affect the efficiency and rate of the phototoxicity.

At 404, the process 400 includes recovering the cultivation media comprising the one or more cell components.

The various embodiments described above can be combined to provide further embodiments. All of the U.S. patents, U.S. patent application publications, U.S. patent applications, foreign patents, foreign patent applications, and non-patent publications referred to in this specification and/or listed in the Application Data Sheet, including but not limited to PCT Publication No. WO/2003/101197 published Dec. 11, 2003; PCT Publication No. WO/2004/091584 published Oct. 10, 2004 are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for releasing a cell component from a photosynthetic organism, comprising:
contacting the photosynthetic organism with an energy-activatable sensitizer; and
activating the energy-activatable sensitizer, thereby releasing a cellular component from at least one of a membrane structure, tubule, vesicle, cisterna, organelle, cell compartment, plastid, or mitochondrion, associated with the photosynthetic organisms.

2. The method of claim 1, wherein said energy-activatable sensitizer is activatable by absorption of light, sonic, ultrasonic, thermal, and/or chemical energy.

3. The method of claim 1, wherein said cellular components are recovered.

4. The method of claim 1, further comprising:
providing a permeabilizer to the photosynthetic organism, the permeabilizer capable of promoting absorption of the energy-activatable photosensitizer by the photosynthetic organism.

5. The method of claim 4, wherein the permeabilizer comprises polyethylenimine.

6. The method of claim 1 wherein the energy activatable photosensitizer is activatable by absorption of energy having a wavelength in the visible, ultra violet, and/or infrared range.

7. The method of claim 1 wherein the energy activatable photosensitizer is activatable by absorption of energy having a wavelength ranging from about 500 nm to about 1100 nm.

8. The method of claim 1 wherein the energy activatable photosensitizer is activatable by absorption of energy having a wavelength ranging from about 200 nm to about 400 nm.

9. The method of claim 1 wherein the energy activatable photosensitizer is activatable by absorption of energy having a wavelength ranging from about 400 nm to about 780 nm.

10. The method of claim 1 wherein the released cellular components comprise one or more growth factors, amino acids, carotenoids, bioflavinoids, carbohydrates, chlorophylls, enzymes, co-enzymes, fatty acids, lipids, minerals, nucleic acids, pigments, proteins, or vitamins.

11. A system for releasing a cellular component of a photosynthetic organism, comprising:
a bioreactor comprising:
a container having an exterior surface and an interior surface, the interior surface defining an isolated space configured to retain a plurality of photosynthetic organisms and cultivation media, and
a first lighting system comprising one or more energy-emitting substrates received in the isolated space of the container, each having a first surface and a second surface opposite to the first surface, the one or more energy-emitting substrates configured to supply a first amount of energy from the first surface and a second amount of energy from the second surface to at least some of a plurality of photosynthetic organisms retained in the isolated space; and
cultivation media for sustaining a plurality of photosynthetic organisms, the cultivation media comprising at least one photosensitizer;
wherein the first lighting system is operable to selectively emit energy having a peak emission wavelength ranging from about 400 nm to about 780 nm during a first period of time, and operable to selectively emit energy having a peak emission wavelength ranging from about 200 nm to about 400 nm during a second period of time, different than the first.

12. The system of claim 11 wherein the second amount of energy has at least one characteristic that has a value that is different than a value of a characteristic of the first amount of energy.

13. The system of claim 11 wherein the at least one characteristic is at least one of a light intensity, an illumination intensity, an energy-emitting pattern, a peak emission wavelength, an on-pulse duration, and/or a pulse frequency.

14. The system of claim 11 wherein the photosensitizer is energy activatable, and the first lighting system is operable to selectively emit energy having a peak emission wavelength corresponding to an activation energy of the photosensitizer.

15. The system of claim 11 wherein the one or more energy-emitting substrates are configured to supply an effective amount of energy to a substantial portion of the plurality of photosynthetic organisms retained in the isolated space, the effective amount of energy sufficient to cause the at least one photosensitizer to disrupt one or more membrane structures, tubules, vesicles, cisternae, organelles, cell compartments, plastids, or mitochondria, associated with the plurality of photosynthetic organisms.

16. A composition for releasing one or more growth factors, amino acids, nucleic acids, carotenoids, bioflavinoids, carbohydrates, chlorophylls, enzymes and co-enzymes, fatty acids, lipids, minerals, nucleic acids, pigments, proteins, and/or vitamins from an algal biomass into a collection medium, the composition comprising:
a plurality of energy-activatable photosensitizers, the energy-activatable photosensitizers activatable by absorption of light, sonic, ultrasonic, thermal, and/or chemical energy;
a permeabilizer to promote absorption of the energy-activatable photosensitizers by the algal biomass.

17. The composition of claim 16 wherein the algal biomass is selected from a group comprising prokaryotic algae and eukaryotic algae.

18. The composition of claim 16 wherein the algal biomass is selected from one or more micro-algae.

19. A process for producing and recovering one or more cell components from culture media including a plurality of photosynthetic organisms, comprising:
inducing a dielectric change in the culture media, the induced dielectric change sufficient to induce photo-oxidative stress of a substantial portion of the plurality of photosynthetic organisms; and
recovering the cultivation media comprising the one or more cell components.

20. The process of claim 19 wherein recovering the cultivation media comprising the one or more cell components includes chromatographically recovering one or more growth factors, amino acids, carotenoids, bioflavinoids, carbohydrates, chlorophylls, enzymes and co-enzymes, fatty acids, lipids, minerals, nucleic acids, pigments, proteins, and vitamins.

21. The process of claim 19, wherein the plurality of photosynthetic organisms is selected from a group comprising prokaryotic algae and eukaryotic algae.

22. The process of claim 19, wherein the plurality of photosynthetic organisms is selected from one or more micro-algae.
